(19) United States
(12) Patent Application Publication
(10) Pub. No.: US 2021/0219393 A1
(43) Pub. Date: Jul. 15, 2021

(54) VARIABLE VISIBLE-WAVELENGTH LIGHT OUTPUT DEVICES FOR BIOLOGICAL INTERACT AND METHODS OF THEIR USE.

(71) Applicant: Lumen Hygienic, LLC, St. Petersburg, FL (US)

(72) Inventors: James Kerr, Scarborough, ME (US); Stephen Almeida, Tampa, FL (US)

(21) Appl. No.: 16/740,405

(22) Filed: Jan. 11, 2020

(51) Int. Cl. H05B 45/10; A61L 9/20; H05B 45/20

(52) U.S. Cl. CPC H05B 45/10; A61L 2209/12; H05B 45/20; A61L 9/20

(57) ABSTRACT

Disclosed and claimed herein are devices and methods for disease prevention and more specifically in the field of devices and methods for disease prevention by the control of visible or visible/UV light wavelength ranges when they impinge on biological systems and entities.

### REFERENCE TO PRIOR FILED APPLICATIONS

This application claims the benefit of U.S. Provisional Application No. 62/946,291, filed 10 Dec. 2019 under 35 U.S.C. § 119(e).

### FIELD OF THE INVENTION

The present application for patent relates to devices and methods for disease prevention and more specifically in the field of devices and methods for disease prevention by the control of visible or visible/UV light wavelength ranges when they impinge on biological systems and entities.

### BACKGROUND

It is known that blue light, particularly in the wavelength range of 405-470 nm, exhibits antimicrobial effects without the addition of external photosensitizers. It is also known that blue light is much less detrimental to biological cells than is ultraviolet radiation which is also used for antimicrobial applications. It has also been reported that blue light exposure can result in cell-to-cell communication via blue light receptors in bacteria and inhibit biofilm formation and subsequently result in light inactivation and therefore ineffective antibacterial effects.

However, at higher intensities, blue light exhibits a broad-spectrum antimicrobial effect against both Gram-positive and Gram-negative bacteria. Blue light therapy is a clinically accepted approach for Propionibacterium acnes infections. Studies on blue light inactivation of important wound pathogenic bacteria, including Staphylococcus aureus and Pseudomonas aeruginosa have also been reported.

Additionally, as can be seen in the above discussion, the correct amount of blue light exposure can be the difference between effect and ineffective treatment; for some biological entities light which has too high an intensity can deactivate the blue light treatment while for other biological entities low intensity light can be beneficial allowing for increases in growth. Thus, depending on the biological entity, the effectiveness of blue light exposure will depend on the blue light intensity. In addition, the light recommended and/or required in various medical environments have a blue light component, which in the case of some pathogens may aid in their growth causing undesirable effects.

While various strategies have been put forward for fighting diseases, these solutions are starting to be ineffective. For example, buildup of immunity of some microorganisms from repeated treatments, inappropriate and changing prescriptions of antibiotics for viral diseases, the failure of some patients to complete their treatment regimen, and overuse of antibiotics in livestock feedstuffs also exacerbate the problem. Many physicians are concerned that several bacterial infections soon may become untreatable. In addition to its adverse effects on public health, antibiotic resistance contributes to higher health care costs. Treating resistant infections often requires the use of more expensive or more toxic drugs and can result in longer hospital stays for infected patients. As a result, a major research effort has been led to find alternative antimicrobial approaches to which, it is hypothesized, bacteria will not be easily able to develop resistance.

It should be noted that current recommendations for light in hospitals is 5000 k with strong emissions in the 400-496 nm range, FIG. 3. The issues relating to this recommendation will become apparent in the body of this disclosure.

Thus, there is a long desired and unmet need for alternate strategies to combat diseases, infection and anti-microbial solutions. The current disclosure discusses devices and methods of their uses to address these issues.

### BRIEF DESCRIPTION OF THE DRAWINGS

FIG. 1 displays examples of wavelength outputs of a number of commercially available LEDs useful in the current disclosure.

FIG. 2 displays examples of the basic chemical makeup of commercially available LEDs useful in the current disclosure.

FIG. 3 displays the recommended wavelength output for hospital lighting

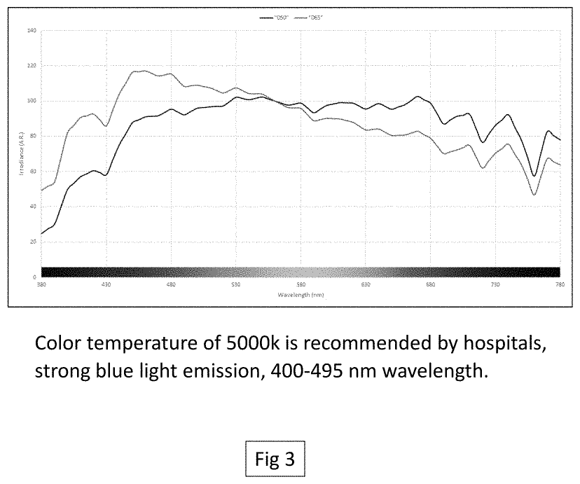

FIGS. 4 A and B show the effect blue light has on various microorganisms

FIG. 5 shows a representation of the device of the current disclosure in operation

### SUMMARY OF EXEMPLARY EMBODIMENTS

Disclosed and claimed herein are devices and methods for disease prevention. The devices are comprised of an array of LEDs which emit in the visible light spectrum. UV emitting LEDs many also be included. The LEDs are controlled by a central processing unit (CPU) which raises and lowers the intensity of one or more of the LEDs. The CPU is preprogrammed to change the output intensities of the LEDs to levels that are associated with the effect the wavelength and intensity the light has on the disease or biological entity being addressed. Additionally, methods of using the devices include preprogramming the CPU with light intensity data associated with certain diseases and biological entities and using a control panel or wireless device or both to communicate with the CPU to deliver the correct signals to the LEDs to provide the desired intensity of the LEDs.

In a first embodiment, disclosed and claimed herein is a device comprising an array of LEDs, each LED having a specific range of wavelength outputs determined by the LED chemical make-up and an intensity output, wherein each individual LED is configured to be adjustable in its intensity output.

In a second embodiment, disclosed and claimed herein is the device of the above embodiment wherein the specific range of wavelength outputs are chosen from the wavelengths of the visible light spectrum and the ultraviolet light spectrum.

In a third embodiment, disclosed and claimed herein are any of the devices of the above embodiments further comprising a central processing unit (CPU) in electronic communication with the LEDs and is configured to electronically adjust the intensity output of each of the individual LEDs when a command is given to the CPU

In a fourth embodiment, disclosed and claimed herein are any of the devices of the above embodiments wherein the central processing unit is programmed to adjust the intensity output of each LED to a predetermine intensity output level which has been chosen based on the interaction of the specific wavelength ranges and the specific wavelength range intensity outputs with chosen target biological entities.

In a fifth embodiment, disclosed and claimed herein are any of the devices of the above embodiments wherein the intensity outputs are adjustable from 0% to 100% intensity output levels.

In a sixth embodiment, disclosed and claimed herein are any of the devices of the above embodiments wherein the chosen wavelength range outputs and the chosen wavelength intensity output levels are detrimental or beneficial to the chosen target biological entities.

In a seventh embodiment, disclosed and claimed herein are any of the devices of the above embodiments wherein the biological entities are viruses, bacteria, pathogens, microorganisms, living cells or a combination thereof.

In an eighth embodiment, disclosed and claimed herein are any of the devices of the above embodiments wherein the chosen wavelength range outputs and the chosen wavelength intensity output levels are detrimental or beneficial to the immune system of the biological entities.

In a ninth embodiment, disclosed and claimed herein are any of the devices of the above embodiments further comprising a control panel in communication with the central processing unit whereby the control panel is configured to input instructions to the central processing unit for adjustment of the chosen levels of wavelength ranges and intensity levels of the LEDs.

In a tenth embodiment, disclosed and claimed herein are any of the devices of the above embodiments wherein the central processing unit is configured to be controlled by wireless communication.

In an eleventh embodiment, disclosed and claimed herein are any of the devices of the above embodiments wherein the device is configured to be mobile and/or handheld.

In a twelfth embodiment, disclosed and claimed herein are any of the devices of the above embodiments wherein the specific range of wavelength outputs are chosen from the wavelengths of the ultraviolet light spectrum.

In a thirteenth embodiment, disclosed and claimed herein are any of the devices of the above embodiments wherein the device is configured to be an ambient light source.

In a fourteenth embodiment, disclosed and claimed herein are any of the devices of the above embodiments wherein the device is configured to be a permanent light source.

In a fifteenth embodiment, disclosed and claimed herein are any of the devices of the above embodiments wherein the devices and method are configured for use in hospitals, veterinarian hospitals, emergency rooms, operation rooms, waiting rooms, hospital bedroom, ICUs, NICUs, natal and prenatal applications, in the home, sick beds, nurseries, bathrooms, kitchens, area where people, animals, or other biological organism congregate and cause the proliferation of undesirable microorganisms.

In a sixteenth embodiment, disclosed and claimed herein are any of the devices of the above embodiments wherein the device is a ceiling light, a bulb, an elongated lamp, a table lamps, a recessed light fixture, a light which is used to shine directly onto a wound, skin, or other areas of interest of a biological entity.

### DETAILED DESCRIPTION

As used herein, the conjunction “and” is intended to be inclusive and the conjunction “or” is not intended to be exclusive unless otherwise indicated. For example, the phrase “or, alternatively” is intended to be exclusive.

As used herein, the term “biological entity” is any germ, bacteria, virus, disease organism, microorganism, pathogen, living cell or other organism.

As used herein, the term “wavelength range” includes ranges from 1 nm to 300 nm

As will become evident, various modifications and enhancements of the above embodiments are within the scope of the subject matter disclosed and claimed herein.

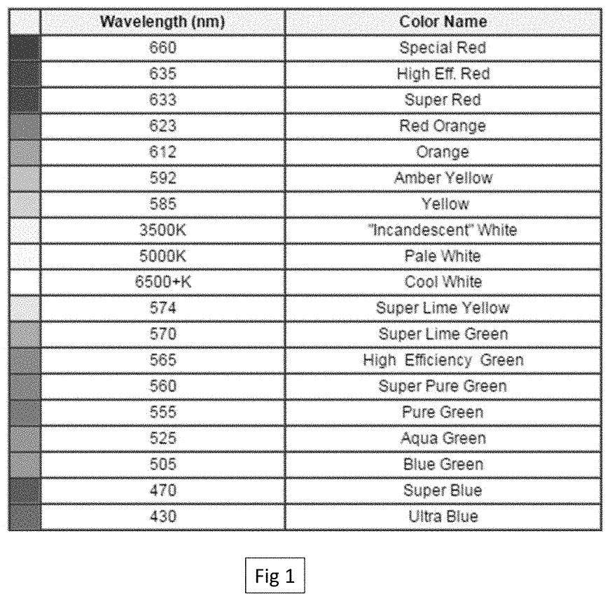

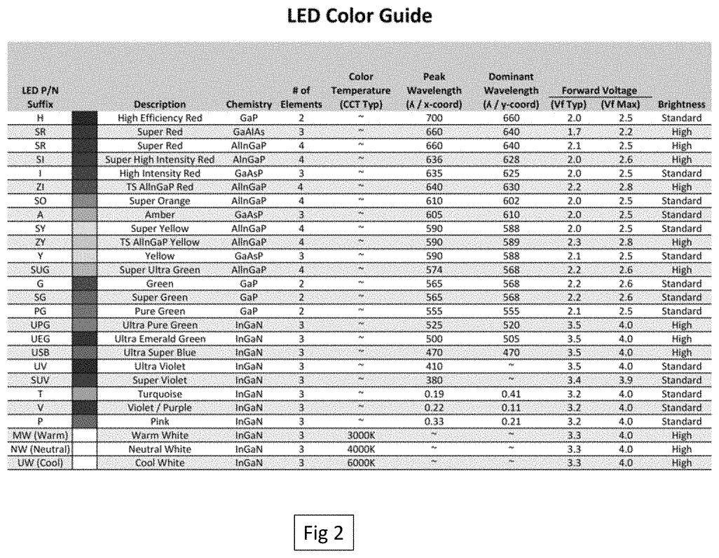

Disclosed and claimed herein is a device comprising an array of light emitting diodes (LEDs). The array may be arranged in any number of geometric patterns, such as, for example, a row, a number of rows, a circular pattern, an oval pattern, layers of patterns and the liked. The array may consist of three or more LEDs, depending on the desired wavelength output and resultant total spectrum output of the LEDs when they are in use. Each LED has a specific range of wavelength outputs determined by the LED chemical make-up and an intensity output. The wavelength range may range from a few nanometers to a few hundred nanometers. FIG. 1 displays examples of wavelength outputs of a number of commercially available LEDs useful in the current disclosure. Any and all of the LEDs can be used in the array of the current disclosure, chosen to provide a desired, combined spectral output. FIG. 2 displays examples of the basic chemical makeup of commercially available LEDs useful in the current disclosure. One can obtain any wavelength, with its associated range, from an LED by changing the composition and materials used in the manufacture of the semiconductor from which the LED is made. Essentially if you know the wavelength you want, you can calculate back to determine the bandgap the semiconductor needs to be in order to achieve the wavelength range according to the following formula: Wavelength=1240.8/Bandgap (in eV).

The LEDs of the current disclosure are configured to allow adjustments to the intensity of each LED. Tunable LED wavelength output is well known, as described in: https://www.energy.gov/eere/ssl/understanding-led-color-tunable-products

Note that the current technology changes the basic color of the output, for example, from blue to red and colors in between. The current disclosure reduces or eliminates wavelength ranges, for example, only wavelengths from 440-480 nm, while the remaining wavelengths continue to irradiate. In this manner the object, room or facility will remain lit so that various activities may continue unabated. Also, the color of the emitted light may not be perceptibly changed.

The devices of the current disclosure further contain a central processing unit (CPU) in electronic communication with the LEDs and is configured to electronically adjust the intensity output of each of the individual LEDs. The LEDs of the current disclosure are configured so that the intensity output of the LED can be adjusted. There are many ways known on the art to adjust the intensity output of an LED, such as, for example, Pulse Width Modulation for controlling the power output using a microprocessor of a CPU, a specialized variable-current circuit or other control component that controls the current passing through the LED. The intensity of the LEDs may be adjusted from 100% to 0% output. In this manner, selected wavelength bands or ranges of wavelength bands may be eliminated from the output spectrum of the LED array. As well, other LEDs may have their output adjusted to reach maximum power output. In this manner the LED array can provide any range or ranges of light output including wavelengths and intensities.

The wavelength output from the LEDs of the current disclosure may be from the visible spectrum and the ultraviolet spectrum so that the LEDs array may emit only visible light, only ultraviolet light or a combination of both. The intensity of the LEDs are adjusted depending on the target biological entity, including visible intensity, UV intensity or both.

The devices of the current disclosure contain a central processing unit (CPU) which is programmed to adjust the intensity output of each LED to a predetermine intensity output level, anywhere from 0% output to maximum output each. A command is received by the CPU from devices, such as for example, a control panel which is electronic communication with the CPU, a control panel which is in wireless communication with the CPU and the like. The CPU is in electronic communication with the LED allowing the intensity output to be adjusted. The program can be altered to add new settings as new research finds new light conditions that effect the biological entities. In this manner the devices can be kept up to date with the latest findings.

The devices of the current disclosure further contain a control panel which is in electronic communication with the CPU which in turn is in electronic communication with each of the LEDs. The panel inputs instructions to the CPU and the CPU send signals to adjust the LED intensity output. The panel may have preset touch areas for adjusting the LED output for commonly known biological entities. Additionally, the CPU may receive wireless instructions from a wireless device, the CPU then, again in electronic communication with the LEDs, sends an electronic signal to adjust the LED intensity levels.

In operation, the chosen wavelength range outputs and the chosen wavelength intensity output levels are either increased or decreased, such as to 0% intensity or 100% maximum intensity or any intensity in between. It is known that some wavelengths of light, both UV and visible, can be detrimental or beneficial to particular biological entities. The CPUs of the devices of the current invention are programmed to adjust the LEDs intensity outputs to address the specific biological entity. For example, if pathogen A thrives in yellow light, the control panel electronically signals the CPU to adjust the LED intensity output to be reduced or eliminated in the yellow region of the output spectrum. The reduction could affect one or more LEDs that output the particular wavelength bands that cause pathogen A to thrive. Conversely, a beneficial microorganism may flourish when exposed to blue light. Thus, the control panel will send an electronic signal to the CPU to adjust the LED intensity output to be increased or maximized in the blue region of the output spectrum. The increase could affect one or more LEDs that output the particular beneficial wavelength bands.

For example, the programmed output may be determined to eliminate wavelength ranges that help to proliferate various pathogens, such as, for example, Brucella, the bacteria that cause the infectious disease brucellosis. It has been recently found that when exposed to the blue wavelengths of sunlight, the signaling proteins in Brucella signal other proteins that tell the bacterium to reproduce at will. In lab experiments, samples were shielded from blue light and compared to controls illuminated with regular white light. If the regular light was strong enough, Brucella grew rapidly and became infectious. When shielded from blue light, the reproductive rate dropped by more than 90%. The devices of the current disclosure would be used to reduce or eliminate the blue segment of the spectrum when Brucella is present.

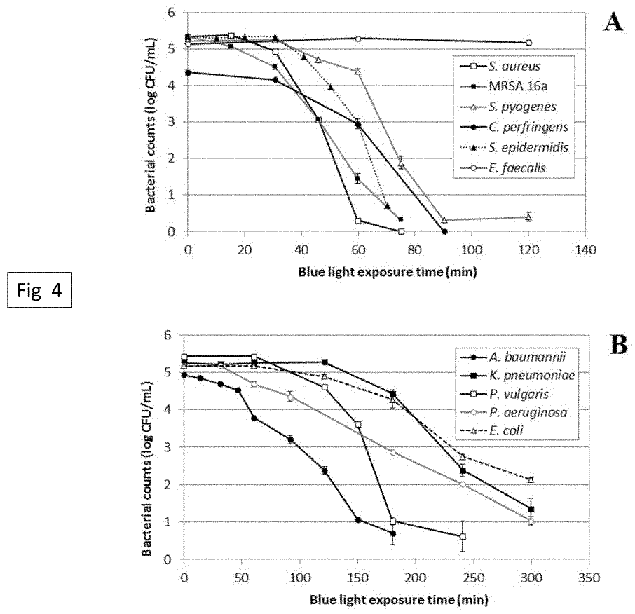

It is also known that visible light at high intensity can kill bacteria that are frequently found in infected wounds while low-power white light enhances bacterial proliferation. The phototoxic effect was found to involve induction of reactive oxygen species (ROS) production by the bacteria. ROS production following blue (400-500 nm) light illumination was found to be higher than that of red (500-800 nm). Within the blue range, light of 415 nm induced more ROS than 455 nm, which correlated with results obtained for the reduction in colony count of S. aureus and E. coli following illumination using equal intensities of these two wavelengths. At low fluencies, both 415 and 455 nm enhanced proliferation of S. aureus but reduced viability of E. coli. Thus, the devices of the current disclosure would be programmed to receive a command from a control panel to control the blue light output of the LEDs. Shown in FIG. 4 are graphs of the decrease in bacterial counts of a number of microorganism when exposed to blue light over time. For example, in FIG. 4B E. coli is reduce by over 60% when exposed to blue light for 5 hours, while in FIG. 4A S, aureus is eliminated when exposed to blue light for 70 minutes.

In other embodiments of the current disclosure, the devices may be used for the illumination of living cells, either to benefit the cells or to be detrimental to the cell.

In another embodiment the devices and methods of the current disclosure are configured to positively affect the immune system of biological entities. It is well known to those of skill in the art that exposure to light of various wavelengths when directed to the eye and the retina of an animal, such as a human, can have beneficial effects on the immune system. The current device may be programmed to deliver any of a mixture of LED wavelength ranges depending on the beneficial effect desired.

The devices of the current disclosure contain a control panel that is in electronic communication with a CPU. The CPU is in electronic communication with each of the LEDs to alter the intensity of the energy output. As above the ability to adjust the intensity output of an LED is well known. The CPU of the current disclosure is programmed to alter the intensity output of the LEDs to levels that are associated with a particular viruses, bacteria, pathogens, microorganisms, living cells or a combination thereof, or a target immune response, or a desired mammalian response.

The devices of the current disclosure are configured so that new instructions may be programmed into the CPU when new findings and information are found wherein the current disclosure may be used.

The devices of the current disclosure may each have a dedicated control panel, or a control panel may provide instructions to the CPUs for more than one device. For example, a bank of 8 LED arrays may be controlled by one control panel situated at particular position in a room such as at the door of the room.

The devices of the current disclosure may be in electronic communication with a main central processing unit that provides electronic instructions to all the devices in a particular environment such as, for example, a hospital in which a central computer controls all the devices in the hospital.

The devices of the current disclosure may be stationary such as, for example, in a hospital room, operating room, waiting room, hospital bed, home or office, or they may be mobile so that the devices may be wheeled, carried or otherwise transported from area to area. The devices of the current disclosure may also be hand-held.

The devices of the current disclosure may be controlled by way of a control panel that is in electronic communication with the CPU or they may be configured to be controlled wirelessly or a combination of both.

The devices of the current disclosure may also contain LEDs which emit in the UV range, either along with LEDs that emit in the infrared range or alone. These devices may be configured to be ambient light sources useful, for example, in public environments, office environment and the like, wherein certain wavelength band outputs are reduced, eliminated or increased, as determined to benefit the occupants of the environment This application extends to other biological species, such as, for example, animals in veterinarian environments, lab testing facilities, microbiology labs and environments, each of which require control of the biological activity associated with the discipline.

The devices of the current disclosure are applicable to a number situations, some of which are noted above, but also include hospitals, veterinarian hospitals, emergency rooms, operation rooms, waiting rooms, hospital bedroom, ICUs, NICUs, natal and prenatal applications, in the home, sick beds, nurseries, bathrooms, kitchens, area where people, animals, or other biological organism congregate and cause the proliferation of undesirable microorganisms.

The devices of the current disclosure may be configured to be ceiling light, a bulb, an elongated lamp, a table lamps, a recessed light fixture, or a light which is used to shine directly onto a wound, skin, or other area of a biological entity.

The devices of the current disclosure may contain a rechargeable power pack to power the device, or they may be hard wired into the electrical serve or have a removal plug.

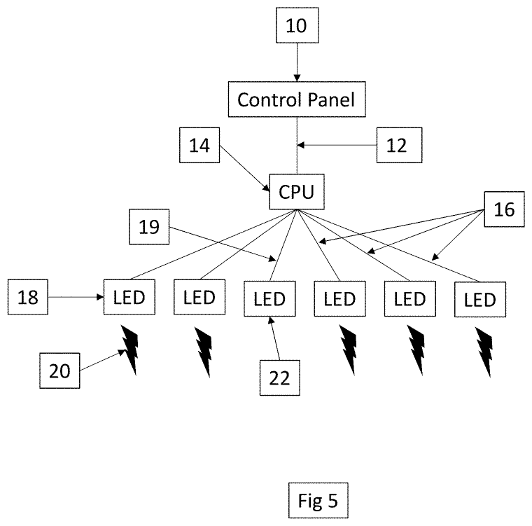

FIG. 5 shows a representation of devices of the current disclosure in operation. The control panel 10 sends an electronic signal 12 to the CPU 14. The CPU 14 sends signals 16 (3 are shown) to the LEDs 18 (1 is shown) which output light 20. Note that one signal 19 is sent to LED 22 where the signal 19 does not activate the LED.

Although the present invention has been shown and described with reference to particular examples, various changes and modifications which are obvious to persons skilled in the art to which the invention pertains are deemed to lie within the spirit, scope and contemplation of the subject matter set forth in the appended claims.

We claim:

1. A device comprising an array of LEDs, each LED having a specific range of wavelength outputs determined by the LED chemical make-up and an intensity output, wherein each individual LED is configured to be adjustable in its intensity output.

2. The device of claim 1, wherein the specific range of wavelength outputs are chosen from the wavelengths of the visible and ultraviolet light spectrum.

3. The device of claim 2 further comprising a central processing unit in electronic communication with the LEDs and is configured to electronically adjust the intensity output of each of the individual LEDs when a command is given to the central processing unit.

4. The device of claim 3, wherein the central processing unit is programmed to adjust the intensity output of each LED to a predetermine intensity output level which has been chosen based on the interaction of the specific wavelength ranges and the specific wavelength range intensity outputs with chosen target biological entities.

5. The device of claim 4, wherein the intensity outputs are adjustable from 0% to 100% intensity output levels.

6. The device of claim 5, wherein the chosen wavelength range outputs and the chosen wavelength intensity output levels are detrimental or beneficial to the chosen target biological entities.

7. The device of claim 6, wherein the biological entities are viruses, bacteria, pathogens, microorganisms, living cells or a combination thereof.

8. The device of claim 5, wherein the chosen wavelength range outputs and the chosen wavelength intensity output levels are detrimental or beneficial to the immune system of the biological entities.

9. The device of claim 6, further comprising a control panel in electronic communication with the central processing unit whereby the control panel is configured to input instructions to the central processing unit for adjustment of the chosen levels of wavelength ranges and intensity levels of the LEDs.

10. The device of claim 6, wherein a control panel is in electronic communication with the central processing unit of more than one of the devices from a remote position, whereby the control panel is configured to input instructions to the central processing units for adjustment of the chosen levels of wavelength ranges and intensity levels of the LEDs.

11. The device of claim 6, wherein the devices are in electronic communication with a main central processing unit.

12. The device of claim 6, wherein the central processing unit is configured to be controlled by wireless communication.

13. The device of claim 6, wherein the device is configured to be mobile.

14. The device of claim 6, wherein the specific range of wavelength outputs are chosen from the wavelengths of the ultraviolet light spectrum.

15. The device of claim 6, wherein the device is configured to be an ambient light source.

16. The device of claim 6, wherein the device is configured to be a permanent light source.

17. The device claim 6, wherein the devices and method are configured for use in hospitals, veterinarian hospitals, emergency rooms, operation rooms, waiting rooms, hospital bedroom, ICUs, NICUs, natal and prenatal applications, in the home, sick beds, nurseries, bathrooms, kitchens, area where people, animals, or other biological organism congregate and cause the proliferation of undesirable microorganisms.

18. The device claim 6, wherein the device is a ceiling light, a bulb, an elongated lamp, a table lamps, a recessed light fixture, a light which is used to shine directly onto a wound, skin, or other area of a biological entity.

19. The device of claim 6, further comprising a rechargeable power pack to power the device.
